(19) United States
(12) Patent Application Publication
(10) Pub. No.: US 2001/0031784 A1
(43) Pub. Date: Oct. 18, 2001

(54) Crystalline base of citalopram

(76) Inventors: Hans Petersen, Vanlose (DK); Klaus Peter Bogeso, Horsholm (DK); Per Holm, Vanlose (DK)

Correspondence Address: DARBY & DARBY P.C., 805 Third Avenue, New York, NY, 10022, US

(21) Appl. No.: 09/730,490

(22) Filed: Dec. 5, 2000

(30) Foreign Application Priority Data
Mar. 13, 2000 (DK) PA 2000 00402

(51) Int. Cl. A61K 31/34

(52) U.S. Cl. 514/469; 549/462

(57) ABSTRACT

The crystalline base of the compound citalopram, 1-[3-(dimethylamino)propyl]-1-(4-fluorophenyl)-1,3-dihydro-5-isobenzofurancarbonitrile, having the formula

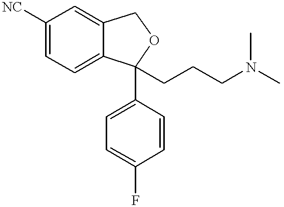

and its use in a process for the manufacture of a citalopram salt is described.

The present invention relates to the crystalline base of the well known antidepressant drug citalopram, 1-[3-(dimethylamino)propyl]-1-(4-fluorophenyl)-1,3-dihydro-5-isobenzofuran-carbonitrile, formulations of said base, a process for the preparation of salts of citalopram, such as the hydrobromide, using the base and the purified salts obtained.

### BACKGROUND OF THE INVENTION

Citalopram is a well-known antidepressant drug that has now been on the market for some years and has the following structure;

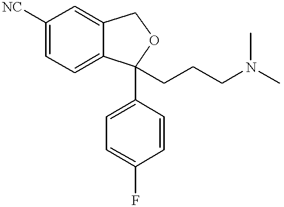

It is a selective, centrally acting serotonin (5-hydroxytryptamine; 5-HT) reuptake inhibitor, accordingly having antidepressant activities. The antidepressant activity of the compound has been reported in several publications, e.g. J. Hyttel, Prog. Neuro-Psychopharmacol. &Biol. Psychiat., 1982, 6, 277-295 and A. Gravem, Acta Psychiatr. Scand., 1987, 75 , 478-486. The compound has further been disclosed to show effects in the treatment of dementia and cerebrovascular disorders, EP-A-474580.

Citalopram was first disclosed in DE 2,657,271, corresponding to U.S. Pat. No. 4,136,193. This patent publication describes the preparation of citolopram by one method and outlines a further method, which may be used for preparing citalopram. The citalopram prepared was isolated as the oxalate, the hydrobromide and the hydrochloride salt, respectively. Furthermore, the citalopram base was obtained as an oil (B.P. 175 C/0.03 mm/Hg). Citalopram is marketed as the hydrobromide and the hydrochloride, respectively. No further forms of citalopram than the above mentioned have been disclosed.

A number of processes for the preparation of citalopram have been disclosed. In many of these the last step of the process is a conversion of a group different from cyano in the 5 position of the direct analogue of citalopram to a 5-cyano group. So citalopram has been prepared by:

Exchange of 5-halogen with cyano (DE 2,657,271 and co-pending PCT/DK 9900643 and PCT/DK 99009640)

Conversion of a 5-amido or 5-ester group to a 5-cyano group (WO 9819513)

Conversion of a 5-amino group to a 5-cyano group (WO 9819512)

Conversion of a 5-formyl group to a 5-cyano group (WO 9900548)

Conversion of a 5-oxazolinyl or 5-thiazolinyl group to a 5-cyano group (co-pending PCT/DK 990576)

Other processes for the preparation of citatopram comprise exchange of the 5-bromo group of 1-(4-fluorophenyl)-1,3-dihydro-5-isobenzofuranbromide with 5-cyano followed by alkylation with a 3-(N,N-dimethylamino)propyl-halogenide (DE 2,657,271 and WO 9819511).

Many of the processes mentioned above have the disadvantage that it is difficult to separate the intermediates formed during the process (the intermediates mentioned above or earlier intermediates) formed the end product and, accordingly, extensive purification procedures are required in order to obtain the necessary quality of the end product.

It has now been found that the base of citalopram may be obtained as very nice and pure crystalline product, which may easily be handled and conveniently be formulated into tablets and other pharmaceutical forms. Furthermore, it has surprisingly been found that a very good and efficient purification of citalopram is obtained during manufacture of citalopram (e.g. of the hydrobromide or the hydrochloride salt) by setting free and crystallising the base.

### SUMMARY OF THE INVENTION

The present invention provides the crystalline base of the compound

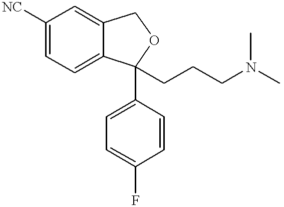

In a second aspect the invention provides a process for the manufacture of a salt of citalopram, preferably the hydrobromide or hydrochloride in which the free base of citalopram is precipitated in crystalline form and then transferred to a pharmaceutically acceptable salt of citalopram.

In a further aspect the invention relates to the pure crystalline salt, preferably the hydrobromide or hydrochloride prepared by the process of the invention.

In yet another aspect, a pharmaceutical formulation of the free base is provided. Preferably the formulation is for oral administration.

The crystalline base of citalopram is preferably more than 99.8% w/w pure, most preferably more than 99.9% w/w (peak area). The melting point is preferably a range within (DSC; onset, open capsule) 90-93° C., most preferably 91-92° C. or it is between 92 and 94° C., preferably 92.5 and 93.5° C. (DSC; onset, closed capsule). In particular it is the crystalline base of the racemic mixture of citalopram.

The base may be set free from a crude salt crude salt or from a crude mixture comprising the free base. The crude salt may be any convenient salt, such as the hydrobrode, hydrochloride, sulphate, oxalate, phosphate, nitrate or any other convenient salts, preferably the hydrobromide or hydrochloride salt. Other salts are salts of organic acids.

The terms crude salt and crude mixture refers to the fact that the salt and the mixture, respectively, comprise impurities, which must be removed or which it is desired to remove. The crude salt may be a salt separated directly from the reaction mixture, or it may have been subjected to some initial or simultaneous purification, e.g. one re-crystallisation, treatment with activated carbon or silica gel. This salt may be prepared by any of the above-mentioned processes and it might be obtained directly by the reaction or it may be formed subsequently by treatment with an acid. The salt may be isolated by precipitation or it may exist in a solvent, e.g. in the mixture resulting directly from the synthesis of the compound. Similarly the crude mixture comprising citalopram base may be obtained directly from the synthesis of the compound according to any of the above mentioned processes or it may have been subjected to some initial or simultaneous purification, e.g. one re-crystallisation, treatment with activated carbon or silica gel.

The base of citalopram may be set free from the crude salt by dissolving the crude salt in a mixture of water and an organic solvent and then adding a base. Alternatively it may be isolated from a crude mixture of the base by purification and extraction. The organic solvent may be toluene, ethyl acetate or any other suitable solvent and the base may be any convenient base, preferably NaOH or NH₃. The base of citalopram is collected by separation of the organic phase, evaporation of the solvent in order to obtain the base most probably as all oil and then crystallisation of the base from an aprotic solvent, such as an alkane, including n-heptane, hexane and isooctane, and high and low boiling petroleum ethers and substituted aromates, incl toluene and xylenes.

The pharmaceutically acceptable salt of citalopram, such as the hydrobromide or hydrochloride, may be prepared by methods known in the art. So, the base may be reacted with either the calculated amount of acid in a water miscible solvent, such as acetone or ethanol, with subsequent isolation of the salt by concentration and cooling, or with an excess of the acid in a water immniscible solvent, such as ethylether, ethylacetate or dichloromethane, with the salt separating spontaneously. The hydrobromide or hydrochloride of citalopram obtained by the method of the invention has a very high purity, preferably more than 99.8% pure, most preferably more than 99.9% purity. Other salts of citalopram, e.g. the oxalate, may also be obtained in a very pure form by this process.

The pharmaceutical compositions of the invention may be administered in any suitable way and in any suitable form, for example orally in the form of tablets, capsules, powders or syrups, or parenterally in the form of usual sterile solutions for injection.

The pharmaceutical formulations of the invention may be prepared by conventional methods in the art. For example, tablets may be prepared by mixing the active ingredient with ordinary adjuvants and/or diluents and subsequently compressing the mixture in a conventional tabletting machine. Examples of adjuvants or diluents comprise: Corn starch, potato starch, talcum, magnesium state, gelatine, lactose, gums, and the like. Any other adjuvant or additive colourings, aroma, preservatives etc. may be used provided that they are compatible with the active ingredients.

Solutions for injections may be prepared by solving the active ingredient and possible additives in a part of the solvent for injection, preferably sterile water, adjusting the solution to the desired volume, sterilisation of the solution and filling in suitable ampoules or vials. Any suitable additive conventionally used in the art may be added, such as tonicity agents, preservatives, antioxidants, etc.

According to the present invention the base of citalopram has been found to be crystalline with stable and nice white crystals and it has been found that the base may easily be crystallised in a very pure form. So for example more than 99.8% w/w pure citalopram base was obtained by crystallisation from up to 95% pure bydrobromide without further purification. Accordingly, the process of the invention for preparing salts of citalopram has been found to give the salts as very pure products of pharmaceutically acceptable quality. Accordingly, the yield of citalopram may be improved substantially during the manufacture of citalopram by avoiding one or more conventional re-crystallisation steps.

Finally, it has been found that the base may be formulated into very good and stable solid formulations with good release properties.

The invention is further illustrated by the following examples.

### EXAMPLE 1

Crystallisation of R,S-Citalopram as the free base.

1-(3-Dimethylaminopropyl)-1-(4′-fluorophenyl)-1,3-dihydrobenzofuran-5-carbonitrile.

1-(3-Dimethylaminopropyl)-1-(4′-fluorophenyl)-1,3-dihydrobenzofuran-5-carbonitrile hydrobromide (101 grams, 0.25 mole) is suspended in water (500 ml) and toluene (500 ml). NaOH (60 ml, 5 N (aq)) is added and the mixture (pH>10) is stirred for 15 min. before the phases are separated. The organic phase is washed with water (2×100 ml) and filtered through a pad of filter help. The volatiles are removed in vacuo and the title compound is obtained as an oil. n-Heptane (400 ml) is added and the mixture is heated to 70° C. On cooling crystals forms. The white crystals of the title compound are filtered off and dried at ambient temperature over night in vacuo. Yield: 75.4 grams (93%). DSC (onset, open capsule): 91.3-91.8° C. DSC (onset, closed capsule): 92.8° C. Purity: (>99.8% (peak area)). Anal. calcd. for C20H21N2F1O1; C, 74.04; H, 6.54; N, 8.64. Found C, 74,01; H, 6.49; N, 8.59. 1H-NMR (DMSO-d6, 500 MHz): 1.21 (1H, m), 1.29 (1H, m), 2.02 (6H, s), 2.09-2.23 (4 H, m), 5.15 (1H, d J=12.5 Hz), 5.22 (1H, d J=12.5 Hz), 7.16 (2H, t J=8.5 Hz), 7.60 (2H, dt J=8.5 Hz J=1.2 Hz), 7.76 (1H, d J=8.5 Hz), 7.79 (1H, d J=8.5 Hz), 7.80 (1H,s), 13C—NMR (DMSO-d6, 125 MHz): 21.8, 38.3, 45.0, 58.8, 71.0, 90.7, 110.5, 115.1 (d J=22 Hz), 118.8, 123.1, 125.1, 127.0 (d J=8 Hz), 132.0, 140.0 (d J=3 Hz), 140.5, 149.5, 161.3 (d J=245 Hz).

### EXAMPLE 2

a) A crude mixture of Citalopram and sulphuric acid is made basic by adding NaOH and the citalopram base is extracted with toluene. The toluene is evaporated and the citalopram base obtained is dissolved in n-heptane at elevated temperature. The very pure free base of citalapram is precipitated by cooling.

b) A crude mixture of Citalopram and sulphuric acid is made basic by adding NaOH and the citalopram base is extracted with toluene. The toluene is evaporated and the citalopram base obtained is dissolved in methanol. The mixture is treated with activated carbon and filtrated and the solvent is evaporated. The purified tree base is dissolved in n-haptane at elevated temperature. Then the very pure free base of citalopram is precipitated by cooling.

c) A crude mixture of Citalopram and sulphuric acid is made basic by adding NaOH and the citalopram base is extracted with toluene. The toluene phase is treated with silicagel, the toluene is evaporated and the citalopram base obtained is dissolved in n-heptane at elevated temperature. The very pure free base of citalopram is precipitated by cooling.

d) A crude mixture of Citalopram and sulphuric acid is made basic by adding NaOH and the citalopram base is extracted with toluene. The toluene phase is treated with silicagel, the toluene is evaporated and the citalopram base obtained is dissolved in methaol. The mixture is treated with activated carbon and filtrated and the solvent is evaporated. The purified free base is dissolved in n-heptane at elevated temperature. Then the extremely pure free base of citalopram is precipitated by cooling.

### EXAMPLE 3

Wet granulation and preparation of tablets

The batch size was 200 g and the granulation was performed in a small-scale laboratory high shear mixer (Micromixer).

Citalopram base was sieved through a sieve aperture of 0.3 mm. The ingredients of the intragranular phase (1-4 in Table 2) were mixed at 600 rpm. 25 ml of purified water (5) was added in 30 sec and the granulation terminated after a total processing time of 3 min. The granulate was wet sieved through a 0.7 mm sieve aperture and dried at 40° C. in 30 minutes to equilibrium relative humidity of 32%. The dried granulate was finally sieved through a 0.7 mm sieve aperture.

The dried granulate was mixed for 3 minutes with the extragranular phase (6-7) in a Turbula mixer and finally mixed with the lubricant (8) for 30 sec.

| Materials | % |
|---|---|
| Citalopram (base) | 16.00 |
| Kollidon VA64 | 2.32 |
| Lactose 350 mesh | 38.98 |
| Corn starch | 20.00 |
| Purified water | 25 |
| Avicel PH 200 (Microcrystalline cellulose) | 20.00 |
| Ac-Di-Sol (Croscarmelose sodium) | 2.00 |
| Magnesium stearate | 0.7 |

Table 2. Composition of the tablets.

Tablets were produced on a single punch tabletting machine Korsch EKO. The characteristics of the tables are shown in Table 3.

TABLE 2
Tablet characteristics.

| Parameter | Values |
|---|---|
| Tablet strength, mg | 20 |
| Nominal tablet weight, mg | 125 |
| Tablet diameter, mm | 7 |
| Tablet shape | Film coating |
| | Special doomed |
| Mean disintegration time, min | 1.77 |
| Mean chrushing strength, N | 69.1 |
| Mean tablet weight, mg | 125.4 |
| RSD tablet weight, % | 0.42 |
| Friability, % | 0.3 |

The tablets produced had satisfactory technical properties.

### EXAMPLE 4

Melt granulation

The batch size was 200 g. Citalopram base was sieved through a sieve aperture of 0.3 mm.

The granulation was performed in a small-scale laboratory high shear mixer Micromixer).

The ingredients of the intra-granular phase (1-3 in Table 6) were mixed at 1200 rpm. The jacket temperature was 80° C. The granulation process was terminated after 3.5 min. The granulate was sieved through a sieve aperture of 1.0 mm and mixed with the extra-granular phase (4, 5) for 3 min. and with the lubricant (6) for 30 sec.

TABLE 3
Composition of the tablet.

| Materials | % |
|---|---|
| Citalopram (base) | 16.00 |
| Polyethyleneglycol 6000 | 9.14 |
| Lactose 350 mesh | 38.98 |
| Avicel PH 200 (Microcrystalline cellulose) | 30.00 |
| Kollidon CL (Cross-linked povidone) | 4.00 |
| Magnesium stearate | 0.7 |

Tablets were produced on a single punch tablettig machine Korsch EKO. The characteristics of the tables are shown in Table 4.

TABLE 4
Tablet characteristics.

| Parameter | Values |
|---|---|
| Tablet strength, 20 mg | 20 |
| Nominal tablet weight, mg | 125 |
| Tablet diameter, mm | 7 |
| Tablet shape | Film coating |
| | Special doomed |
| Mean disintegration time, min | 1.0 |
| Mean chrushing strength, N | 55.5 |
| Mean tablet weight, mg | 125.6 |
| RSD tablet weight, % | 0.5 |
| Friability, % | 0.4 |

The tablets produced had satisfactory technical properties.

1. A Crystalline base of the compound citalopram, 1-[3-(dimethylamino)propyl]-1-(4-fluorophenyl)-1,3-dihydro-5-isobenzofurancarbonitrile, having the formula

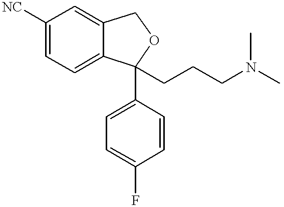

2. The crystalline base according to claim 1, characterised in that it is the base of the racemic mixture of citalopram.

3. The crystalline base according to claim 1 or 2, characterised in that it has a melting point of 90-93° C., most preferably 91-92° C.

4. The crystalline base according to any of claims 1-3, characterised in that is has a purity of more than 99.8% w/w, preferably more than 99.9% w/w.

5. A process for the manufacture a salt of citalopram characterised in that the base of citalopram is set free and precipitated in crystalline form and then transferred to the salt.

6. The process of claim 5 for the manufacture a salt of citalopram characterised in that the base of citalopram is set free from a crude salt of citalopram and precipitated in crystalline form and then transferred to the salt.

7. The process according to claim 5 or 6, characterised in that the salt prepared is the hydrobromide or hydrochloride salt of citalopram.

8. The process according to claim 6, characterised in that the crude salt is a hydrobromide, hydrochloride, sulphate, oxalate, phosphate or nitrate salts, preferably the sulphate hydrobromide or hydrochloride salt.

9. The process of claim 5 characterised in that the base of citalopram is set free from a crude solution of citalopram base or salt.

10. The hydrochloride or hydrobromide salt of citalopram prepared by the process of claim 5, 6, 7, 8 or 9.

11. The hydrochloride or hydrobromide salt of citalopram of claim 10, characterised in that it has a purity of more than 99.8% w/w, preferably more tan 99.9% w/w.

12. A pharmaceutical formulation of the free base of citalopram of any of claims 1-4, preferably tablets or a melt granulate.
